(19) United States
(12) Patent Application Publication
(10) Pub. No.: US 2008/0171959 A1
(43) Pub. Date: Jul. 17, 2008

(54) Compression Article, Particularly A Compression Stocking With A Surface Pattern

(76) Inventor: Karl Achtelstetter, Dasing-Wessiszell (DE)

Correspondence Address: Fleit Gibbons Gutman Bongini & Bianco PL, 21355 EAST DIXIE HIGHWAY, SUITE 115, MIAMI, FL, 33180, US

(21) Appl. No.: 11/908,644

(22) PCT Filed: Jan. 21, 2006
(86) PCT No.: PCT/EP2006/000537

§ 371 Date: Nov. 5, 2007

(30) Foreign Application Priority Data
Mar. 15, 2005 (DE) 202005004275.7

(51) Int. Cl. A61F 13/08

(52) U.S. Cl. 602/62; 66/178 A; 66/178 R; 2/239

(57) ABSTRACT

The invention relates to a compression article, particularly a compression stocking or compression tights, made of an elastic knitted fabric (1) with a base knitted fabric (2), which is made from a knitting thread (S) and inside of which compression-imparting compression threads (K) are deposited. In order to obtain a surface pattern, the manner in which the compression threads (K) are deposited alternates area by area.

### FIELD OF THE INVENTION

The invention relates to a compression article, particularly a compression stocking according to the preamble of claim 1.

### BACKGROUND OF THE INVENTION

Compression articles, such as, for example, compression stockings, compression tights, or compression bandages, are manufactured normally from an elastic knotted fabric with a base knitted fabric made from a knitting thread. Such compression articles, when worn on the body of the user, apply a blood circulation promoting compression pressure on the tissue lying underneath it. Compression stockings and tights are used, for example, in case of venous diseases, particularly varicose veins, to improve the venous blood flow to the heart and to eliminate venous congestion.

In comparison to fashionable tights for women, compression stockings have an unsatisfactory appearance, and as a result, women in particular do not like to wear such compression stockings.

The invention is now based on the problem of making available more aesthetically pleasing compression articles that can be worn gladly with fashionable clothes.

### SUMMARY OF THE INVENTION

With a compression article made of an elastic knitted fabric with a base knitted fabric which is made from a knitting thread, and inside of which compression-imparting compression threads are deposited, this problem is solved according to the invention in that the compression article presents a more aesthetically pleasing surface pattern, which is also generated by a comparatively varied deposition of the compression threads in the base knitted fabric.

In a preferred embodiment example of the compression article according to the invention, the compressing threads are deposited, for example, in a first area of the knitted fabric offset 1:1, and in the abutting area ribbed 1:1.

### BRIEF DESCRIPTION OF THE DRAWINGS

The invention is explained in greater detail below with reference to the accompanying drawing and an embodiment example. In the drawing, the figures show:

FIG. 1: a schematic representation of the mesh pattern of the knitted fabric from which the compression article according to the invention is manufactured; and

FIG. 2: a schematic representation of the surface pattern of the knitted fabric of FIG. 1.

### DETAILED DESCRIPTION OF THE INVENTION

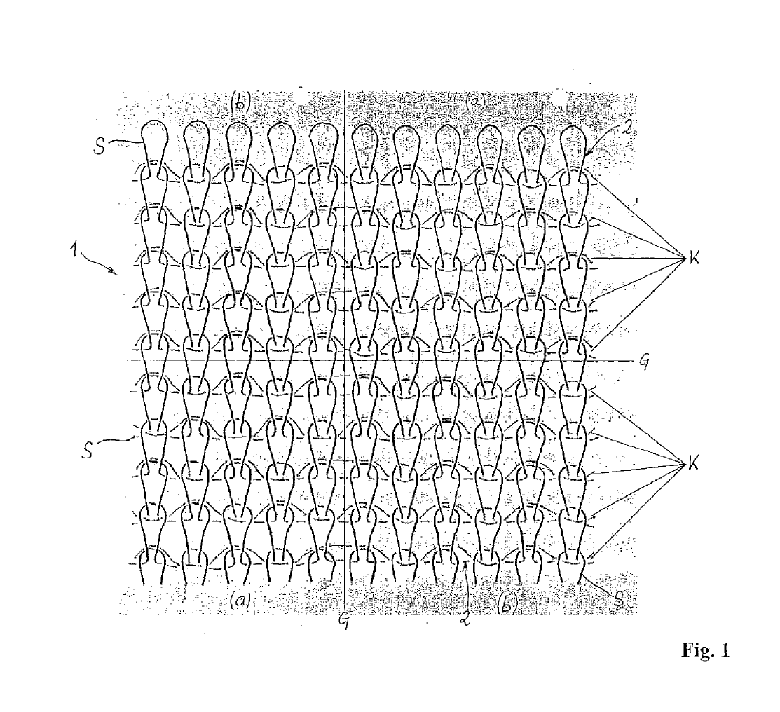

FIG. 1 shows the mesh pattern of a knitted fabric, from which the compression article according to the invention, particularly a compression stocking, compression tights or compression bandage, can be manufactured. The elastic knitted fabric 1 is formed from a base knitted fabric 2 made from a knitting thread S, where the compression-imparting compression threads K are deposited in the base knitted fabric. The knitted fabric 1 shown in FIG. 1 presents a chessboard surface pattern, in which the different areas forming the chessboard pattern a, b, which are arranged adjacently in the manner of a chessboard, produce in each case a different visual impression, so that a chessboard surface pattern becomes apparent. In the section from the knitted fabric 1, which is shown in FIG. 1, a total or four such areas a, b of the chessboard pattern are shown. In the areas a, which are arranged in the section shown on the top right and the bottom left in FIG. 1, the compression threads K are deposited with a 1:1 offset in the base knitted fabric which is in the form of a right/left knitted fabric. In the areas b, which abut the former area [the base knitted fabric], the compression threads K are deposited in the right/left base knitted fabric 2 in a different way, namely with 1:1 ribbing, as is apparent from FIG. 1. The change in the deposition type from 1:1 offset in area a to 1:1 ribbed in area b takes place along the boundary lines G. These boundary lines G can—as shown in FIG. 1—extend either longitudinally, i.e., parallel, to the rows of the base knitted fabric 2, or diagonally with respect to the latter.

The compression threads K are elastofibers, particularly elastodiene, elasthane or elasthane threads wound with polyamide. The knitting threads S are preferably an elasthane thread wound with polyamide. The knitted fabric 1 consists, for example, of 50-70% polyamide and the rest elasthane.

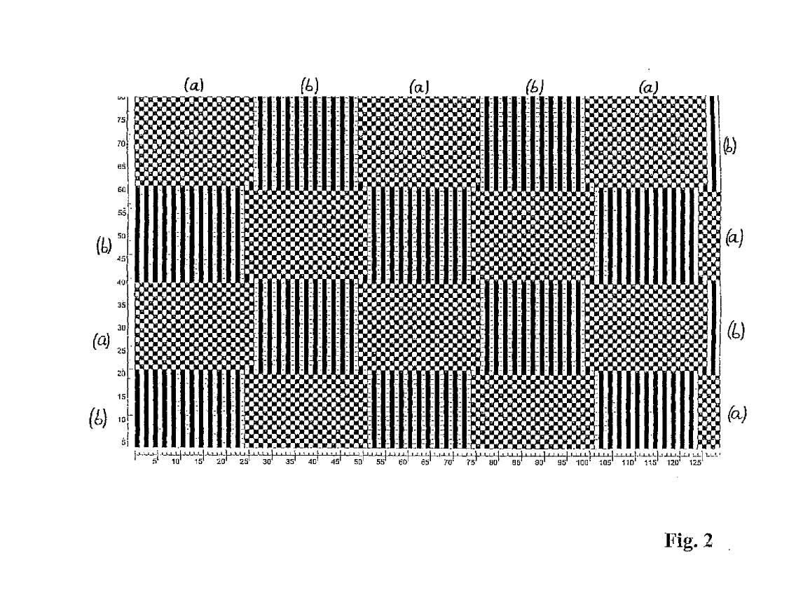

As a result of the difference in the deposition of the compression threads in the different areas, surface patterns of a great variety of geometries can be produced, for example, chessboard, stripe, argyle, bubble, or circle patterns. FIG. 2 is, for example, a schematic representation of a chessboard pattern with the areas a and b alternating in the form of a chessboard. In the latter, the black squares in each case show a compression thread K which is deposited above the mesh head of a mesh of the base knitted fabric 2, and the white squares represent a compression thread K that is deposited at the base of a mesh (see FIG. 1). In the areas a, because of the type of deposition (1:1 offset), a chessboard-shaped inner pattern is represented in the area a, which is graphically represented both in the horizontal and also in the vertical direction as an alternating deposition of the compression threads K in the meshes of the base knitted fabric 2. In the areas b abutting the tatter area, in contrast, a stripe-shaped internal pattern is generated, on the basis of which, in these areas b, compression threads K having the same shape converge toward each other between the different rows of the base knitted fabric 2.

Using such a knitted fabric, compression articles can be manufactured with a great variety of aesthetically pleasing surface patterns. By the simultaneous selection of threads with pleasing colors or color combinations, it is also possible in this way to produce visually pleasing compression articles, which can also be worn with fashionable clothes.

1. Compression article, particularly compression stocking or compression tights, made of an elastic fabric (1) with a base knitted fabric (2), which is made from a knitting thread (S), in which compression-imparting compression threads (K) are deposited, characterized in that, to achieve a surface pattern, the type of deposition of the compression threads (K) is changed in different areas.

2. Compression article according to claim 1, characterized in that the compression threads (K), in a first area (a) of the knitted fabric, are deposited with 1:1 offset, and in an area (b) abutting the former area, they are deposited with 1:1 ribbing.

3. Compression article according to claim 1, characterized in that the base knitted fabric (2) is a right/left knitted fabric.

4. Compression article according to claim 1, characterized in that the base knitted fabric (2) is a round knitted fabric.

5. Compression article according to claim 1, characterized in that the compression threads (K) are elastofibers, particularly elastodiene, elasthane, or elasthane threads wound with polyamide.

6. Compression article according to claim 1, characterized in that the boundary lines (G) are located between the areas (a; b) with a different type of deposition of the compression threads (K) both along and also transverse to the mesh rows of the base knitted fabric (2).

7. Compression article according to claim 1, characterized in that the surface pattern is a chessboard, stripe, argyle, bubble, or circle pattern.
